(19) United States
(12) Patent Application Publication
(10) Pub. No.: US 2016/0278694 A1
(43) Pub. Date: Sep. 29, 2016

(54) Device for Visual Vein Location

(71) Applicants: Abraham Aharoni, Rehovot (IL); Anatoli Rapoport, Rehovot (IL)

(72) Inventors: Abraham Aharoni, Rehovot (IL); Anatoli Rapoport, Rehovot (IL)

(21) Appl. No.: 15/081,765

(22) Filed: Mar. 25, 2016

(30) Foreign Application Priority Data
Mar. 29, 2015 (IL) 237995

(51) Int. Cl. A61B 5/00; A61B 5/06; A61B 1/04; A61B 1/00; A61M 5/32

(52) U.S. Cl. CPC A61B 5/489; A61B 1/00165; A61M 5/3287; A61B 5/6848; A61B 1/00045; A61B 1/04; A61B 5/065; A61B 1/00009

(57) ABSTRACT

A hypodermic needle device has disposable and reusable portions. The disposable portion includes a hypodermic needle having a tubular bore, at least one optical imaging waveguide extending along the needle so that a distal end of the optical imaging waveguide is proximate the tip of the needle, and a first coupler for securing the optical imaging waveguide within the needle at the proximal end thereof. A second coupler remote from the first coupler removably secures a proximal end of the optical imaging waveguide to the reusable portion. The reusable portion includes a mating coupler to removably connect to the second coupler and a respective viewing device so that a user can view the image transmitted by the optical imaging waveguide from its distal end to its proximal end. The second coupler contains a lens for imaging an enlarging the image transmitted from the distal end of the optical imaging waveguide.

### CROSS-REFERENCE TO RELATED APPLICATIONS

The present application claims priority to Israeli Application No 237995 filed on March 29, 2015.

### STATEMENT RE: FEDERALLY SPONSORED RESEARCH/DEVELOPMENT

Not Applicable

### BACKGROUND

The invention relates to visual vein location systems and in particular to a vein location device for the assistance in the insertion of hypodermic needles and catheters in venipuncture, phlebotomy and intravenous therapy.

Several different vein visualization devices for the non-evasive imaging of subcutaneous veins are known in the art. These are based on one of two main technologies: ultrasonic imaging and optical imaging with red or infrared light. Ultrasonic methods deploy transducers that generate and detect ultrasound, and are scanned mechanically or electronically to generate an image of the back-reflected insonification. Veins are imaged clearly with ultrasound, and ultrasonic imaging systems are suitable for guiding intravenous needles directly into a vein. Nevertheless, ultrasonic imaging systems suffer some practical drawbacks, including a poor image close to the skin surface, relatively high cost and large physical size.

The optical technology is based on the relatively high absorption of red and near infrared (NIR) light in blood. On a forearm, for example, illuminated with red or NIR light, the veins are seen as dark areas over a background of brighter regions of light scattered from surrounding tissue.

While optical illumination technologies offer smaller and less costly systems, the depth at which they can visualize veins is limited by two factors: the relatively strong reflection and scatter off the surface of the skin, and the relatively strong back scatter from subcutaneous tissue. Both of these factors gradually reduce the contrast of image veins, limiting the practical imaging depth possible with this method to a few millimeters. In general the applicability of optical absorption visualization is limited to the detection of veins in depth not exceeding some 6 or 8 mm. This is a serious practical limitation, as for many purposes deeper veins are of interest.

A primary motivation of the present invention is to assist in the insertion of intravenous needles for venipuncture, phlebotomy and intravenous therapy, where fluids are injected into the blood stream or blood samples are extracted. State-of-the-art venipuncture devices comprise an assembly of a rigid hypodermic needle or intravenous (IV) needle and a flexible catheter placed over the needle. The assembly mechanically pierces the skin and tissue and penetrates a vein. Once the assembly is located in the vein, the needle is withdrawn and the flexible catheter is secured inside the vein, serving as a duct for transfer of fluid into the blood stream, or removing blood for testing.

Considering the procedure for insertion of the needle, it is generally divided into two parts. First the person performing the procedure, for example a phlebotomist, searches for a suitable vein. A vein with active blood flow (termed a patent vein), with a relatively large size, and a straight stretch with no bifurcations is sought. In searching for a suitable vein, phlebotomists combines their prior knowledge of anatomy and the location of suitable veins, their visual image of the patient's veins, and often the tactile feedback of a vein to manual pressure applied to it. Obviously the latter two inputs can often be very limited, especially in elderly or obese patients, those with dark skin or patients who have low blood pressure due to dehydration or other medical conditions. The present invention aims to assist a phlebotomist in the second stage—the physical insertion of a needle into the selected vein.

In the second part of a conventional procedure, the phlebotomist inserts a needle into the selected vein. This process involves careful aiming of the needle towards the selected vein and navigating its tip towards the center of the vein's width to overcome situations where the vein flexes away at the contact of the needle (a situation called a rolling vein). Once in contact with the vein, the phlebotomist needs to penetrate the frontal vein wall carefully and avoid reaching the opposite vein wall.

U.S. Pat. No. 5,030,207 discloses a device for indicating when an intravenous needle has entered the vein through the use of a solid fiber optic mounted in the needle for showing visual instantaneous vein entry. The distal end of the fiber optic is polished to be flush with the distal point of the needle.

The fiber optic is sized to have an outer diameter which fills the internal bore of the needle. On contact with the blood in the vein, the polished distal end of the solid fiber collects ambient light filtered by the blood and transmits it through the solid fiber to a magnifying arrangement located at the rear or proximal end of the fiber optic. The user observes immediate vein entry without any blood flow or exposure to blood. Other embodiments utilize the solid fiber optic itself for piercing the tissue, thus eliminating the needle altogether. It is also possible to rely on ambient light that is collected by the magnifying arrangement and directed into the solid fiber to illuminate the tip, or use supplementary illuminators to increase the illumination. The operator is constrained to view the vein through a narrow field of view and must distinguish between relatively small variations in the color of the low light level collected by the small solid fiber's tip and transmitted through the optics of the device.

The system disclosed in U.S. Pat. No. 5,030,207 also appears to require the use of a solid optical fiber, there being no suggestion to use a disposable optical fiber or to illuminate the internal opening of the needle directly without using an optical fiber. The illumination is primarily based on ambient light, although an option for supplement illumination at the distal end of the needle is suggested. The illumination is intended to scatter off the blood in the vein, a portion of which enters the solid optical fiber and appears as a red indication to a user viewing the magnification arrangement. Furthermore, there is no indication of the possibility of using an extended optical fiber to allow a mechanically separate illumination source at a convenient distance.

U.S. Pat. No. 4,311,138 likewise discloses a hypodermic needle adapted to emit light from its distal end to facilitate venipuncture under subdued lighting conditions. The needle is used in conjunction with a portable light source, such as a battery handle and lamp, and includes an optical fiber bundle that transmits light from the lamp to the distal end of the needle. A flexible catheter is releasably mounted on the needle and is adapted to be inserted in the vein after the needle has punctured same and thereafter the needle can be withdrawn from the catheter.

The system disclosed in U.S. Pat. No. 4,311,138 appears to require the use of an optical fiber bundle, there being no suggestion to use a disposable single strand optical fiber or to illuminate the internal opening of the needle directly without using an optical fiber. The illumination source considered is a white light lamp with a limited percentage of the light output being coupled into the fiber. The illumination itself is broad band white light which does not serve to accentuate the location of red-absorbing blood vessels. In addition there are no measures included in the disclosure to ensure that fragments of the optical fiber bundle do not break off and remain within a patient's body.

Another visualization approach disclosed in IL Patent Application No. 228868 corresponding to PCT/IL2014/050883 proposes the use of various methods to generate light along the length of a hypodermic needle to emanate from its tip. Such illumination can then be viewed from a vantage point outside the body either with bear eyes or a camera. This needle-tip-emanating illumination assists in the visualization of subcutaneous veins both in the first phase of venipuncture, the location of a suitable vein as well as in the insertion phase of the procedure. In this second phase the distance of the needle-tip-emanating light from the vein is progressively reduced offering gradually improved visualization of the vein as it is approached, and an abrupt decrease in the illumination intensity on penetration into the vein. The needle-tip illumination is provided for by several options: an optical waveguide is inserted into the needle's bore reaching its tip, an optical waveguide illuminating the internal surface of the needle-bore where the bore itself serves to guide the light onto the tip of the needle; and illumination waveguides incorporated into the wall of the catheter; or use of the catheter wall itself as a waveguide to guide the light onto the tip of the needle. This application also proposes supplying the needle-tip-illumination system in two parts: a disposable part including the waveguide use to transfer light to the needle tip, and a reusable part including the illumination source and any auxiliary devices such as camera's and various sensors. The two portions of the system are coupled by a quick release coupler.

French Patent 2 977 497 and WO2014/029423A1 image the veins to the side of the needle—a region that is normally blocked by the needle itself. This is achieved by introducing an opening in the needle itself through which the view of a vein, typically located to the side of the needle under normal operation, is visible. Alternatively an imaging waveguide with a central bore is provided where the needle is located within the central bore. A further alternative provides for an imaging waveguide incorporated into the catheter surrounding the needle.

### BRIEF SUMMARY

It is an object of the present invention to closely assist the delicate needle insertion procedure by providing an optical image of the location the target vein, especially when the target vein is approached, and indications on the instance of penetration into the front wall of the vein, where further insertion of the needle should be arrested to avoid damage to the opposite vein wall.

It is a further object of the present invention to offer similar advantages for automated intravenous needle insertion devices.

This object is realized in accordance with the invention by a hypodermic needle device having the features of the independent claims.

In accordance with one aspect of the invention there is provided a hypodermic needle device, with a primary purpose of implementing a reusable portion containing the more expensive components that are not in direct contact with the patient, and a disposable portion which is less costly and in direct contact with the patient. The disposable portion of the proposed device comprises:

a hypodermic needle having a tubular bore;

at least one imaging optical waveguide extending along said needle so that the distal ends of said imaging optical waveguides are in proximity to the tip of the needle;

a first coupler for securing the at least one optical waveguide within the needle at the proximal end of the needle; and

a second coupler for removably securing a proximal end of the at least one optical waveguide to the reusable portion of the device.

The reusable portion of the device comprises:

A matching coupler to removably connect to the said second coupler;

a respective viewing device where a user can view the back-projected image directly or where the viewing device contains a camera to monitor the back-projected image and project the image onto a viewing screen; wherein:

the reusable portion of the second coupler contains a lens for imaging the output of the proximal end of the imaging waveguide onto the user's eye or the camera; and

the second coupler is remote from the first coupler.

Another aspect of the present invention relates to incorporation of an opening in the hypodermic needle to improve the field-of-view (FoV) of the imaging waveguide toward the side of the needle. This is particularly important for vein location during the insertion process, as typically the needle is slanted at between 10 and 30° to the vein for near-tangential insertion. The FoV of a state-of-the-art forward-looking imaging fiber bundle is directed from the distal end of the fiber bundle outward and is centered on the optical axis of the fiber bundle. The FoV is also partially blocked by the needle tip itself and therefore does not extend far enough to the side of the needle where the vein is located. In the present invention this difficulty is overcome by cutting an opening in the needle behind its tip allowing the extension of the FoV towards the side of the needle.

Another aspect of the invention relates to the placement of the at least one optical imaging waveguide external to the needle; providing for an imaging waveguide with a central orifice for placing around the needle, and an imaging waveguide incorporated into the catheter. All of these aspects of the invention are designed to improve the peripheral viewing of the imaging waveguide for improved view of the target vein, which is nearly parallel to the needle being inserted.

A further aspect of the invention relates to a motion sensor incorporated on the needle/catheter/optical imaging waveguide assembly, providing for feedback of the rotation of the needle about its axis during the insertion process. Using the motion sensor's input, a software algorithm loaded onto the display processor identifies the attitude of the vein in the received image and is therefore able to identify the approximate location of the vein and the background from where signals can be measured to enhance its contrast and outline of the target vein.

Other aspects of the invention relate to a system defined by the respective independent claims.

The present invention is designed to extend the ability of a user to accurately insert a needle into a vein, even when the vein is at larger depths, and assist in guiding a needle tip to the selected vein. This is accomplished by back-projecting an image of the vicinity of the needle-tip to be viewed by the user. In this manner the vein selected for penetration is viewed as it is approached, such view becoming clearer as the distance to the vein decreases, independently from the depth of the selected vein. This also allows the user to manipulate the needle to hit the vein accurately at its center even if the initial insertion direction of the needle was offset.

Furthermore, the image provided clearly and visually indicates the instant of vein penetration so that overshoot of the needle insertion is prevented. As described in more detail in the following, the proposed device is applicable for use in the needle insertion phase, assisting both manual and automated IV needle insertion operations.

The present invention expands on PCT/IL2014/050883 in that it provides for an imaging waveguide that travels the length of the needle. Such a waveguide is used to provide for an image of the vicinity of the needle tip. This image is projected back along the imaging waveguide and viewed either directly by the operator, or, alternatively monitored with a camera to be projected onto a display screen for the operating convenience of the user.

### BRIEF DESCRIPTION OF THE DRAWINGS

These and other features and advantages of the various embodiments disclosed herein will be better understood with respect to the following description and drawings, in which like numbers refer to like parts throughout, and in which:

FIGS. 1A to 1D show schematically the main components of a prior art needle/catheter assembly and their implementation in the different steps of insertion of the IV needle;

FIGS. 2A to 2E show schematically the main components of the proposed needle/catheter/optical imaging waveguide assembly and their implementation in the different steps of insertion of the IV needle;

FIG. 3A shows the state of the art waveguide field-of-view and FIG. 3B shows an enlarged view through a section A-A′ in FIG. 3A;

FIGS. 3C to 3S schematically show different options for implementation of the needle/catheter/optical imaging waveguide assembly according to the invention; and

FIG. 4A and 4B show schematically a visual vein locator system with a camera and a digital display also incorporating a tri-axis rotation sensor on the proximal end of the needle/catheter/optical imaging waveguide assembly state.

### DETAILED DESCRIPTION

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

In the following description of some embodiments, identical components that appear in more than one figure or that share similar functionality will be referenced by identical reference symbols.

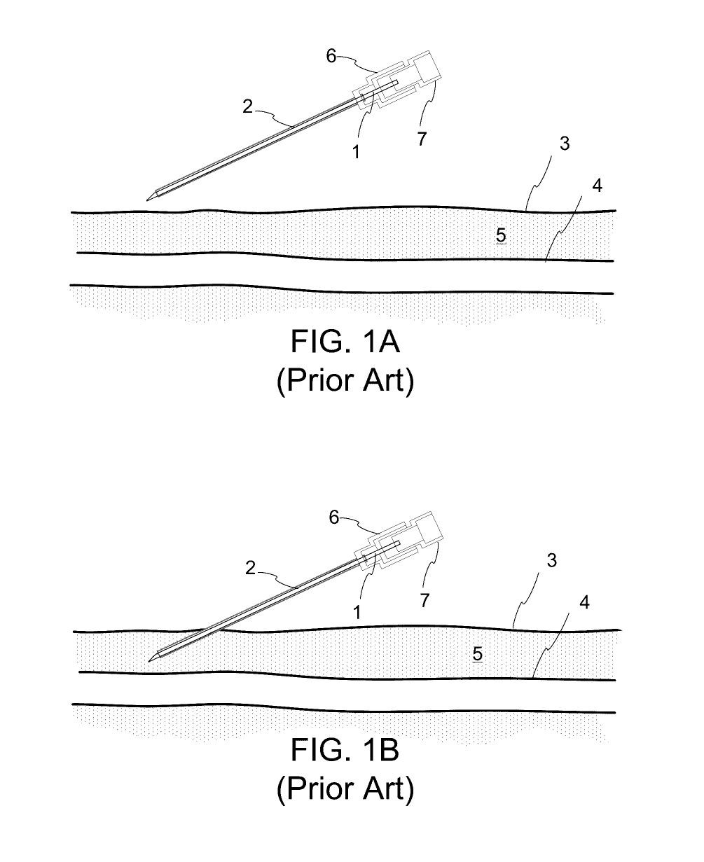

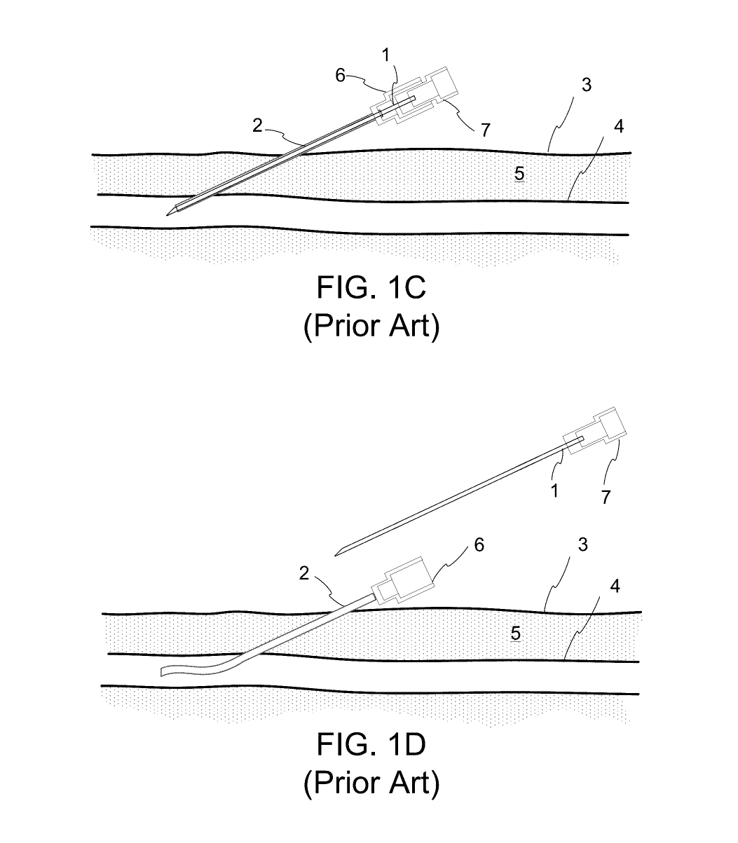

Before describing the invention in detail we consider the state of the art of IV needles and their application shown in FIGS. 1A to 1D. The IV needle assembly comprises a hypodermic penetrating needle 1, and a flexible catheter 2. The assembly is inserted through the skin 3 and into a vein 4 via intervening tissue 5 (FIG. 1B). Typically needles are inserted at an angle of 10 to 30° to the target vein to ensure that penetration of the vein wall is nearly tangential, both for guiding the catheter smoothly into the vein as well as to reduce the risk of puncturing the back wall of the vein. An important feature of the state-of-the-art needles is the backflow of blood that is visible in a suitable chamber at the proximal end of the needle once the vein is penetrated. Such blood backflow indicates that the needle has entered the vein, that further penetrating motion must stop and that care has to be taken so as not to puncture the opposite vein wall. Another important feature is the mechanical interface between the respective interlocking mechanical hubs 6 and 7 of the catheter 2 and needle 1, and which also facilitates attachment to a syringe barrel or other tubing by means of a press-fit or twist-on fitting. Once the catheter 2 is inside the vein 4 (FIG. 1C), the needle can be withdrawn as shown in FIG. 1D. The catheter 2, which is now located inside the vein 4, is used as a duct for either removing blood samples for laboratory testing, or for insertion of plasma or other fluids into the vein.

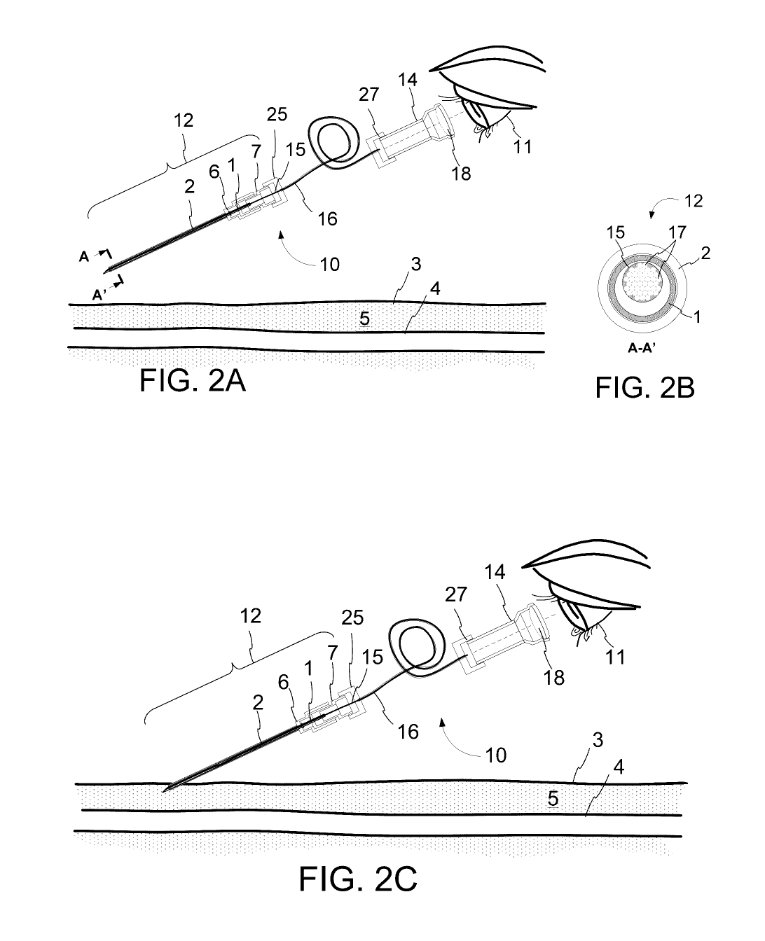

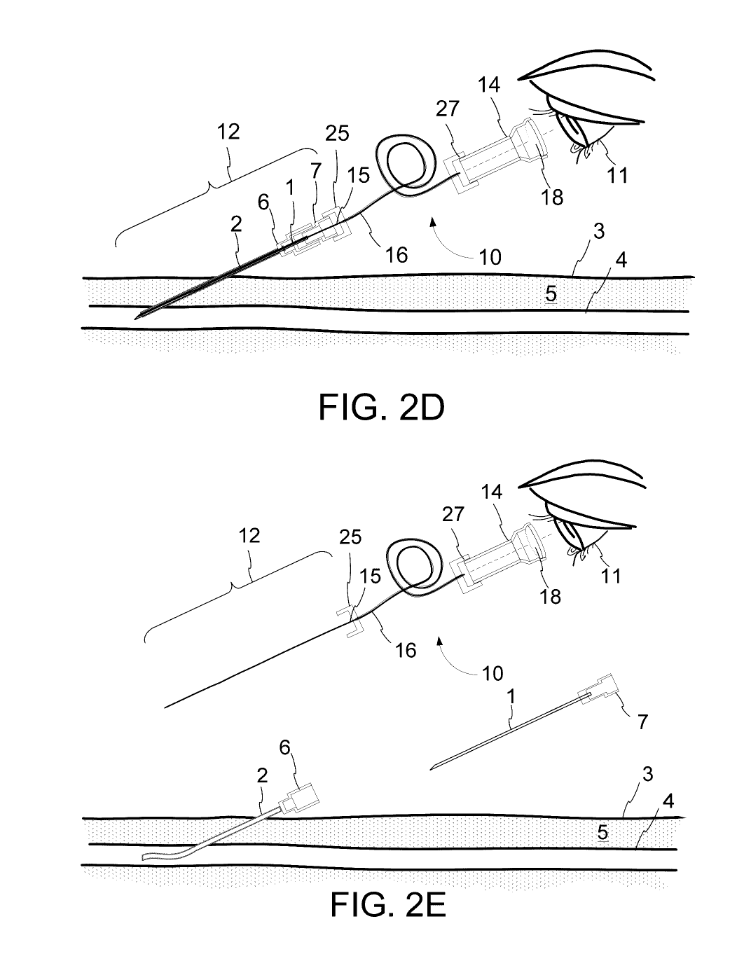

FIGS. 2A to 2E show schematically a hypodermic needle device 10 having a needle/catheter/optical waveguide assembly 12 that is remotely coupled to a viewing optic 14 and its mode of implementation according to an embodiment of the present invention. Similar to FR 2 977 497 and WO 2014/029423 the optical waveguide can be in the form of a fiber bundle 15, comprising an orderly array of individual fibers 17, and may include, in addition to the viewing ability as described above, an illumination coupling into the waveguide to illuminate the viewed target. Alternatively the imaging waveguide may be implemented in the form of a graded index fiber, or a square cross-section optical waveguide: both forms are known to transmit an image at fixed periodic intervals. Additionally, standard, step index fibers, can serve as imaging waveguides. These are known to scramble the transmitted modes and consequently any ordered optical special information, but the scrambling is deterministic and several descrambling methods have been demonstrated allowing image transmission even in such standard fibers. The imaging waveguide 15 may be enclosed within a protective sleeve 16 and is inserted into a state of the art IV needle 1 and flexible catheter 2 assembly such as shown in FIGS. 1A to 1D of the drawings. The needle 1, catheter 2 and the optical imaging waveguide 15 together constitute the needle/catheter/optical imaging waveguide assembly 12. The most commonly used needles for drawing blood for blood tests are 21-gauge needles with inner diameter of 0.514 mm, while the most commonly used needles for blood donation are 16- or 17-gauge needles with internal diameters of 1.194 and 1.069 mm, respectively. Optical fiber bundles with sub millimeter diameter are commercially available, allowing the placement of a fiber inside the needle while leaving sufficient room for blood flow within the internal bore of the needle. Similarly graded index fibers can also be supplied commercially with diameters small enough to fit into any of the above sizes and leave room for backflow of blood as discussed above. Of course, if a standard step index fiber is used with a descrambling technique, these are typically 0.1 mm in diameter and readily fit into any of the above needle sizes leaving ample room from blood flow.

A principal difference between the current device and that of FR 2 977 497 and WO 2014/029423 relates to the connectivity and modularity of the device, and more specifically that the device according to the invention is designed to include a reusable portion and a disposable portion. The disposable portion of the device is that which is in direct contact with the patient, and is generally in contact with the patient's blood. As such, if it were not disposable, it would have to be sterilized. Suitably, the disposable portion of the device also, by design, comprises of low-cost components. Conversely, the reusable portion of the device, is not in contact with the patient, therefore does not require sterilization between applications. It also comprises the more expensive components of the device, such as a camera, processor and display.

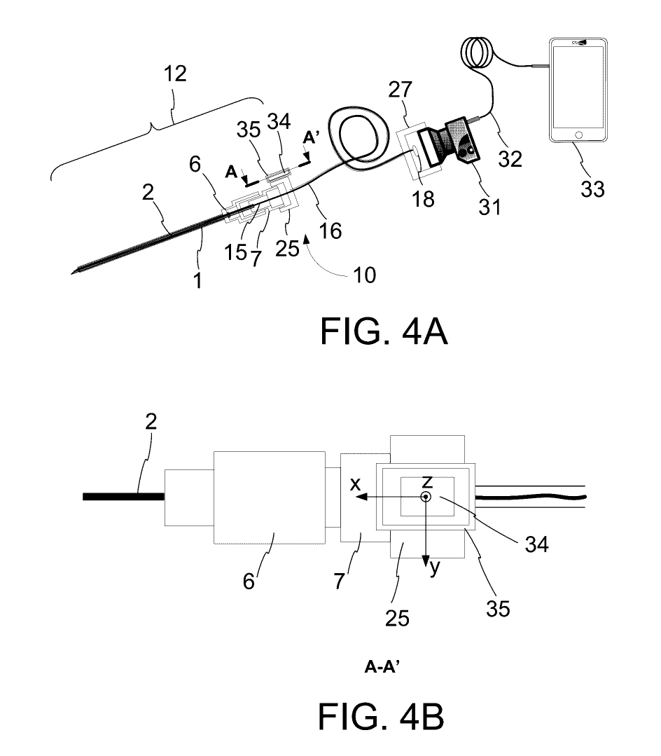

In the disposable portion of the device, the imaging waveguide 15 is secured onto the hub 7 of the IV needle 1 by a suitable distal fitting 25 (constituting a first coupler) using a friction mount or screw mount or other mechanical attachment. The proximal tip of imaging waveguide 15 is mounted inside a removable fitting 27 (constituting a second coupler), which, in turn is connected to the reusable portion of the device comprising a viewing optic 14. The viewing optic 14 can be suitable for direct viewing by the operator, as shown in FIGS. 2A through 2E. Alternatively the viewing optics (FIG. 4A) may include a camera 31, interconnected to an optional processor and a digital display 33 by a suitable cable 32, of the observed image on a display screen. The lens or lenses 18 incorporated within the viewing optic 14 serve to enlarge the image transmitted by the imaging waveguide for convenient viewing.

As seen in FIGS. 2A to 2E, the deployment procedure for the proposed needle/catheter/optical waveguide assembly 12 is similar to that for the state-of-the-art device as shown in FIGS. 1A to 1D. FIG. 2B is an enlarged detail of the assembly cross-section taken along the line A-A′ in FIG. 2A. The assembly is inserted through the skin 3 and tissue 5 (FIG. 2C) into a vein 4 (FIG. 2D). The imaging waveguide within the needle provides a direct of view of the vicinity of the needle tip which is projected back to the viewing optic 14 for monitoring the needle insertion procedure. At larger distances, the image of the vein is “washed out” due to the strong scattering, or turbid nature, of tissue. Nevertheless, similar to viewing an image through fog, as the vein is approached the image of the vein becomes clearer, allowing accurate navigation of the needle-tip to the center of the vein. The image transmitted by the imaging waveguide also provides for clear indication on penetration of the vein, providing a distinct cue for arresting the insertion process to avoid piercing the rear-wall of the vein.

The above description illustrates how the present invention facilitates accurate guidance to a selected target vein, both in the lateral aspect as well as in depth, and provides a distinct indication of the penetration of the front wall of the vein. The latter feature is of primary importance in alerting the person (or robot) inserting the needle to stop moving the needle inward so as to avoid damage to the back wall of the vein. This feature is available in the present invention in addition to the blood backflow on penetration of the vein as occurs in state of the art devices since the arrangement of the imaging waveguide inside the needle leaves sufficient room for blood to backflow.

Once the vein is penetrated and the catheter is in place, the optical imaging waveguide 15 and its assembly can be removed by itself, or together with the needle (FIG. 2E). To remove the imaging waveguide 15 itself, the waveguide's distal fitting 25 is released from the needle hub 6, and the waveguide pulled away and out of the needle. To remove the imaging waveguide and needle together, which as explained below, can both be discarded, the needle is released from the catheter hub 7 as is common to various devices in use. At this point the flexible catheter 2 remains inserted in the vein (FIG. 2E), as with prior art devices, and suitable tubing or other devices, as known in common practice, can be used to remove blood samples or inject fluids. In this respect the present invention does not modify any of the commonly accepted procedures of venipuncture, phlebotomy or intravenous therapy but rather provides for improvements in the location of a target vein while inserting the needle in the insertion phase of the procedure.

It is a primary objective of the present invention to provide for a physically small device that can be handled by a phlebotomist, for example, with essentially no added complexity. Therefore the proposed device is designed as a small addition to state-of-the-art needle/catheter assemblies. In its basic form the needle/catheter assembly is modified only with an additional distal fitting 25. The imaging waveguide 15 and its protective sleeve 16 are small and flexible and essentially do not introduce additional handling difficulty to a phlebotomist. The imaging waveguide 15 can be made sufficiently long to allow the viewing optic 14 to rest at a comfortable distance for manual or screen viewing of the generated image. As described below, some implementations of the present invention do require modifications to the needle/catheter assembly itself. Such changes are not considered to detract from the benefit of the invention since the entire needle/catheter/imaging waveguide assembly 12 may be in the form of a unitary, disposable, sealed, sterilized package, to be opened, ready for use, immediately prior to the insertion of the needle into the vein.

The imaging waveguide, and especially the imaging waveguide's distal region, which is exposed to body fluids of a patient, requires, as a minimum, sterilization. Preferably, the fiber and its supporting parts, referred to as the fiber assembly, can be made disposable, replacing the imaging waveguide's tip with every IV insertion; in this case the imaging waveguide's proximal fitting 27 to the viewing optic is removed and the viewing optics can be reused. Providing for a disposable imaging waveguide assembly offers two practical advantages in addition to the alleviation of the need to sterilize it after every use: as noted above, it can readily be supplied assembled with the needle/catheter assembly in one sterile package to be opened just before use. It is also disposable together with the needle, as described above, so the fiber/needle assembly may be simplified: the imaging waveguide's distal fitting 25 may be molded together with the needle hub 7.

In any case, in one embodiment the present invention provides a personal, pocket-size device that is intended as a personal accessory for medical staff, much like the stethoscope. The personal visual vein locator can serve the phlebotomist in drawing blood tests, nurses and physicians in inserting intravenous catheters in a hospital ward or in the emergency room, as well as paramedics treating injuries in the field. In addition to its small physical size the device is also designed to be low cost, comprising a small number of low cost components: an optical lens viewer, or low cost camera with a common display such as a smart phone, a short optical imaging waveguide and plastic molded casings and tubings. Variations of the personal visual vein locator can be devised as sensors for increasing the automation level of fixtures or automated machinery for replacing various manual operations of the procedure of inserting a needle into a vein.

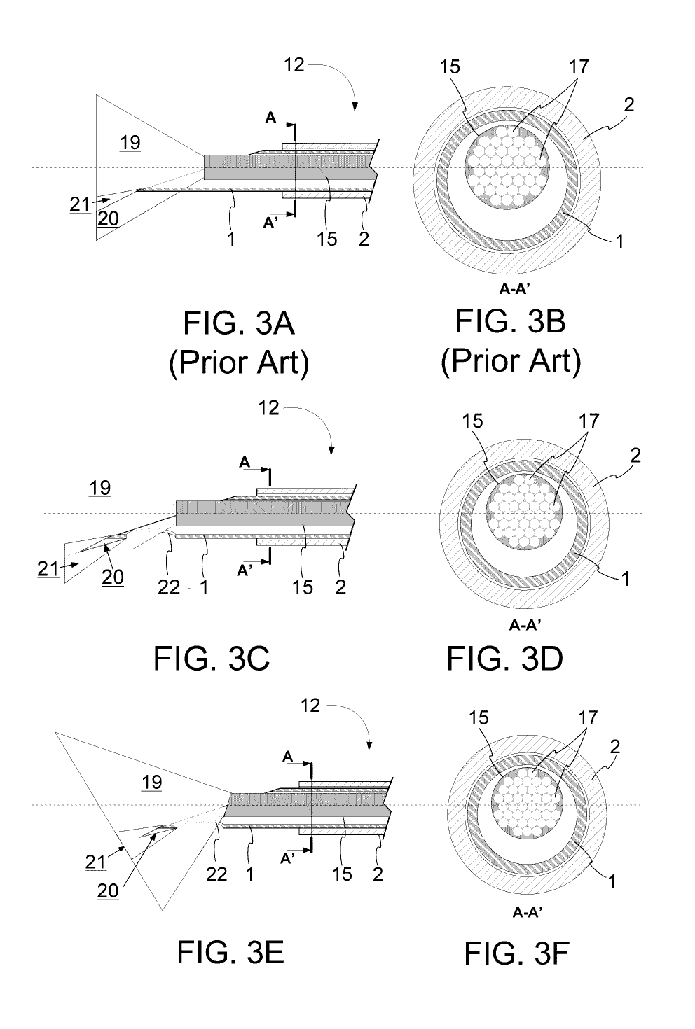

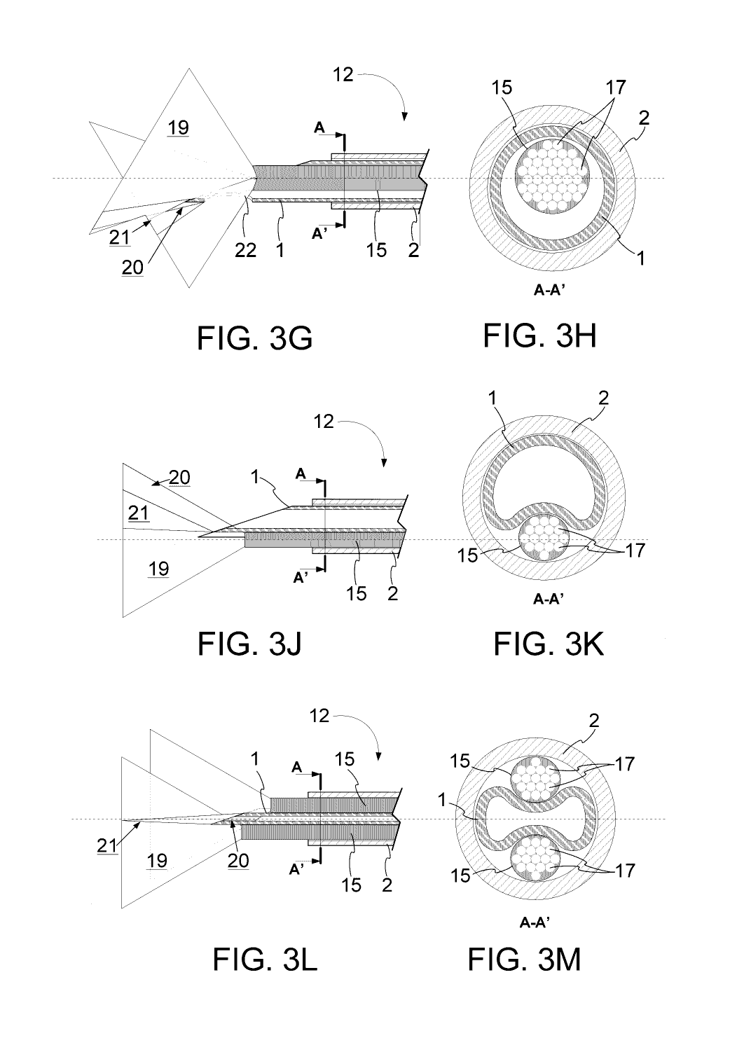

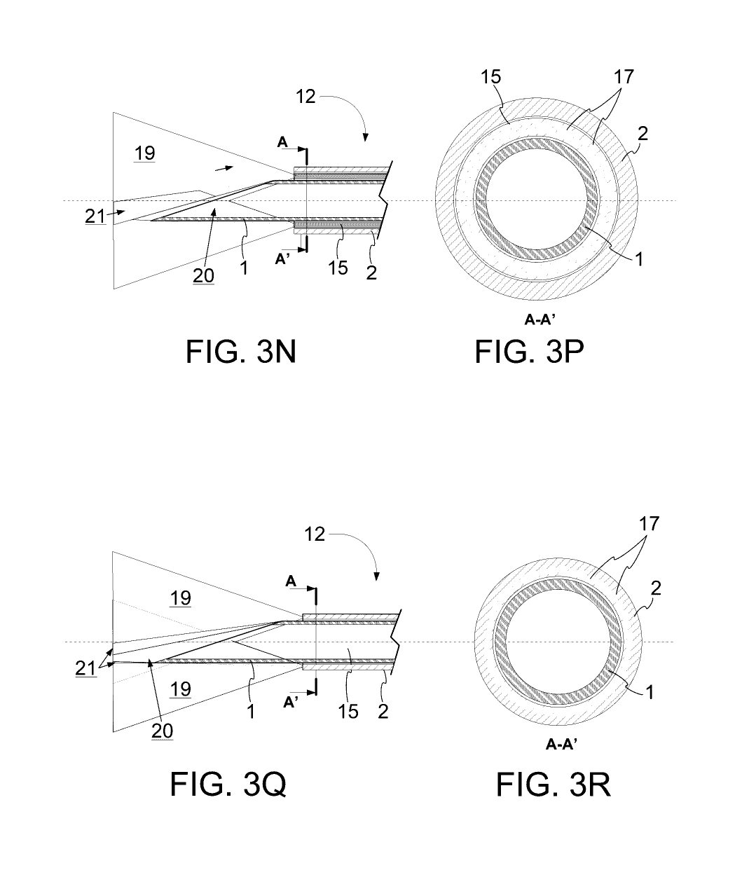

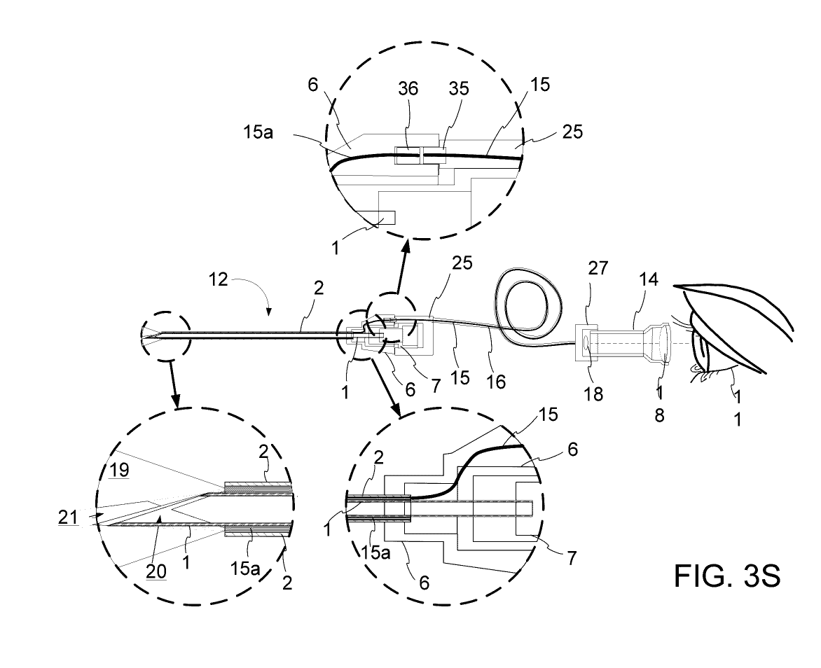

We now consider several modifications to the basic personal visual vein locator described above as depicted schematically in FIGS. 3C to 3S in comparison to the state-of-the-art configurations of FIG. 3A and 3B. For the sake of clarity it is noted that for each configuration, an enlarged cross-section taken along the line A-A′ in the respective figure is shown. By way of example the optical imaging waveguide is depicted in FIGS. 3C through 3S as an imaging fiber bundle 15 with an orderly array of individual fibers 17. As noted above, this is only an example, and the imaging waveguide can be implemented in other forms.

FIG. 3A shows schematically the state-of-the-art situation with an optical imaging fiber bundle 15 inserted into the needle 1; FIG. 3B shows an enlarged cross-sectional view along the line A-A′ in FIG. 3A. As is shown in this image, the primary field-of-view (FoV) of the fiber bundle is directed forward and centered on the mechanical axis of symmetry of the needle. Of the total FoV the upper portion 19 is unobstructed, and there is a portion 21 toward the bottom part of the figure that is completely obstructed by the needle and a portion that is partially obstructed 20. As shown in FIGS. 1A through 1D and FIGS. 2A through 2E, typically IV needles are inserted with their pointed ends closer to the vein, at a small angle to the vein; therefore it is the lower portion of the FoV in FIG. 3A which is more important for the navigation of the needle. Significantly, it is this portion of the FoV which is essentially blocked in prior art devices.

FIG. 3C shows one proposed solution to the blocked FoV. FIG. 3D shows an enlarged cross-sectional view along the line A-A′ in FIG. 3C. Here an opening 22 is cut in the tubular section of the needle behind the needle's tip significantly reducing the portions of the downward directed FoV that are completely blocked 21 and partially blocked 20. This significantly improves the visibility of a vein that is located in that portion of the FoV in a venipuncture procedure. It will be noted that the viewed image is used to guide a needle in the direction of the vein—such an image can tolerate considerable distortions, for example as may occur from a slanted distal imaging waveguide face. An alternative configuration is shown in FIG. 3E, where, in addition to the opening 22 in the needle, the distal face of the imaging waveguide is shaped. FIG. 3F shows an enlarged cross-sectional view along the line A-A′ in FIG. 3E. In the example of FIG. 3E the imaging waveguide's distal face is cut at an angle to refract the light entering the imaging waveguide and tilt the FoV of the image downward in the direction of a target vein. Alternative distal face modifications can be used, including a concave bi-angular distal face (FIG. 3G) for enlarged overall FoV, (FIG. 3H shows the cross-sectional view along the line A-A′ in FIG. 3G) while maintaining the improved downward FoV in the direction of the vein; a convex or concave form for contracting or expanding on the angular spread of the image, and an asymmetric bi-angular distal face for taking a small portion of the image away from the vein as a reference for the background illumination (similar to FIG. 3G, but with the top angle extending to a shorter distance and optionally having a different angle).

An alternative configuration is shown schematically in FIG. 3J, where an imaging waveguide is mounted onto an outer surface of the needle 1, external to it. FIG. 3K shows an enlarged cross-sectional view along the line A-A′ in FIG. 3J. Optionally, the imaging waveguide is located within a suitable recess or groove introduced along the length of the needle as depicted in Section AA′ of the enlarged image of the tip of the needle fiber assembly in FIG. 3J. Such a groove can be formed with a suitable press mould. The imaging waveguide may be optionally cemented to the needle. This arrangement provides for a clear FoV in the direction of a target vein, in that the imaging waveguide is located below the needle (that is in the direction of the target vein) so that the needle does not block the FoV. The distal face of the imaging waveguide may also be angled to tilt the FoV further towards the target vein. Additionally and alternatively, there may be attached to the assembly more than one imaging waveguide for an enlarged overall FoV. FIG. 3L shows a two-imaging waveguide arrangement, of which a magnified section along line A-A′ is show in FIG. 3M. Unlike the implementation of the imaging waveguides central to the needle, the implementation of imaging waveguides external to the needle required coupling through the periphery of the elements 6, 7, and 25.

Another alternative is shown schematically in FIG. 3N of which an enlarged cross-sectional view along the line A-A′ is shown in FIG. 3P. Here a tubular imaging waveguide 15 is inserted around the needle 1 and within the catheter tubing 2. As noted above significant image distortions can be tolerated in this application. The image transmitted through a tubular imaging waveguide would necessarily include image distortions, but there can be tolerated here. The tubular imaging waveguide can be formed as an imaging fiber bundle by preparing a scaled fiber preform and pulling the preform to obtain the smaller dimensions required here. A similar process can be used to form a graded index fiber tubular array as an alternative implementation for such a tubular imaging waveguide. Also, considering a descrambling capability would also allow the implementation of a step-index tubular waveguide.

Yet another alternative is shown in FIG. 3Q where a tubular imaging waveguide is incorporated into the thickness of the catheter, an enlarged cross-sectional view along the line A-A′ being shown in FIG. 3R.

One challenge of the configurations of FIGS. 3J through 3Q relates to the mechanical coupling of the imaging waveguide into the catheter hub 6. One possibility (not shown) is to embed the imaging waveguides into the catheter hub 6 and extend the imaging waveguides continuously to the second coupler 27. This option does not permit the removal of the imaging waveguide assembly from the catheter after the catheter is positioned in the vein. This is inconvenient when the catheter is required for extended operation as, after the catheter is located in the vein, the imaging waveguide is a mechanical disturbance. One possibility to overcome this limitation is to break the imaging waveguide off the catheter hub 6. FIG. 3S shows a more elegant solution where two separate imaging waveguides are provided: one imaging waveguide 15, extends from the second coupler 27 to the first coupler 25 and the other imaging waveguide, 15a, extends from the proximal face of the catheter hub 6 to the annular imaging waveguide 15a in the case of FIG. 3N or the catheter tubing 2 in the case of FIG. 3Q. Mechanical centering and alignment elements are provided: an element 35 mounted onto the first coupler 25 to center and align the distal end of the imaging waveguide connected to the illumination source 15; and an element 36 mounted onto the catheter hub 6 to center and align the proximal end of the imaging waveguide connected to annular imaging waveguide 15a or catheter tubing 2 as required. The mechanical centering and alignment elements ensure, on the one hand, that when assembled the two segments of the imaging waveguides are aligned and the image is transferred efficiently from one imaging waveguide to the other, and, on the other hand, can be separated once the catheter is positioned in the vein. The mechanical elements can be held together with press-fit or twist-on fitting, or with the aid of a breakable pin or latch so that it is possible to manually separate the first coupler 25 from the catheter hub 6.

The image of the target vein delivered through the imaging waveguide becomes clearer as the target fiber is approached. The turbid nature of the tissue decreases the contrast and sharpness of the image with increasing distance from the vein. When a digital image is displayed, this image can be enhanced with image processing procedures. One enhancement can be performed by considering the background illumination away from comparing the target vein and using that as reference for the background thereby allowing the improvement of the contrast of the vein image. This enhancement is particularly important as it alleviates the changes in the target vein image due to variations in the ambient illumination; considering the background where there is no vein serves as a good reference to correctly estimate the location of the vein when the needle is still at a large distance from the target vein and its image is “washed out”. This may be performed by considering the periphery of the image where a vein is not present as reference to the background. Alternatively an asymmetric angle-tilted distal imaging waveguide face provides for imaging of the tissue away from the region of the target vein providing more distinctly the level of the background illumination.

FIG. 4A shows schematically a visual vein locator with a camera 31, processor and digital display 33, wherein a tri-axis rotation sensor 34 is fitted on the needle/catheter/optical imaging waveguide assembly 12 so as to allow the approximate orientation of the target vein to be tracked throughout the needle insertion process. FIG. 4B shows an enlarged cross-sectional view along the line A-A′ in FIG. 4A. As such the area of the background illumination is determined with greater certainty. This serves to provide for a more balanced image of the target vein, even when the needle is still at a relatively large distance from the target vein. In some embodiments the same tri-axis rotation sensor may serve to stabilize the transmitted image, should such a display be required.

The entire discussion above is focused on manual needle insertion procedures, and the visual vein locator is designed to provide a visual display of the target vein through a direct view optics (FIGS. 2A through 2E), or via a camera and digital display (FIGS. 4A and 4B). The very same needle/catheter/optical imaging waveguide assembly with the camera/option can be used to advantage on robotic automated needle insertion machinery. In such cases the image projected by the camera is transmitted to the central robotic system processor where it can be evaluated and serve to close a navigation control circuit that assists the automated needle insertion into the target vein, much in the same process describe above for manual operation.

The description of the above embodiments is not intended to be limiting, the scope of protection being provided only by the appended claims

What is claimed is:

1. A hypodermic needle device, comprising a disposable portion and a reusable portion; the disposable portion comprising:
a hypodermic needle having a tubular bore,
at least one optical imaging waveguide extending along said needle so that a distal end of the at least one optical imaging waveguide is in proximity to the tip of the needle,
a first coupler for securing the at least one optical imaging waveguide within the needle at the proximal end of the needle, and
a second coupler for removably securing a proximal end of the at least one optical imaging waveguide to the reusable portion of the device;
the reusable portion of the device comprising:
a mating coupler to removably connect to the second coupler above;
a respective viewing device in order that a user can view the image transmitted by said optical imaging waveguide from its distal end to its proximal end; wherein:
the second coupler contains a lens for imaging an enlarging the image transmitted from the distal end of the optical imaging waveguide, and
the second coupler is remote from the first coupler.

2. The device as claimed in claim 1, further comprising a camera configured to capture the enlarged image of the least one optical imaging waveguide in the tubular bore of said needle and display it on a screen.

3. The device as claimed in claim 1, wherein the at least one imaging waveguide is an imaging fiber bundle.

4. The device as claimed in claim 1, wherein the at least one imaging waveguide is an imaging graded image fiber.

5. The device as claimed in claim 1, wherein the at least one imaging waveguide is a standard step index fiber, and a descrambling arrangement is further included to allow transmission of images through the step index fiber.

6. The device as claimed in claim 1, wherein the field of view of said at least one optical imaging waveguide is increased by an opening in the needle close to the needle's tip.

7. The device as claimed in claim 1, wherein the distal face of the at least one optical imaging waveguide is shaped to refract the received image.

8. The device as claimed in claim 7, wherein the shape of the distal face of the at least one optical imaging waveguide is slanted to tilt the image field of view with respect to the imaging waveguide's optical axis.

9. The device as claimed in claim 7, wherein the shape of the distal face of the at least one optical imaging waveguide is multiply slanted to expand on the overall field of view of the imaging waveguide.

10. The device as claimed in claim 7, wherein the shape of the distal face of the at least one optical imaging waveguide is curved in two dimensions to vary the acceptance angel of the image.

11. The device as claimed in claim 1, wherein said first coupler is configured to allow removal of the at least one optical fiber from the tubular bore.

12. The device as claimed in claim 1, wherein the at least one optical imaging waveguide is located outside the tubular bore of the needle.

13. The device as claimed in claim 12, wherein the at least one optical imaging waveguide is located in a longitudinal groove within the outer surface of the needle.

14. The device as claimed in claim 1, wherein the at least one optical imaging waveguide is a multi-element imaging waveguide with a tubular cross-section, and where said multi-element imaging tubular waveguide is mounted over and around the needle.

15. The device as claimed in claim 14, wherein the at least one tubular optical imaging waveguide is incorporated into the catheter.

16. The device as claimed in claim 2, further comprising an image processor configured for identifying the background illumination of the transmitted image and enhancing the image contrast of the target vein as viewed through the scattering tissue.

17. The device as claimed in claim 1, further comprising a tri-axis rotation sensor to detect the angular rotation of the image transmitted to the viewing device so as assist in identifying the region of the background in the image.
